SEQUENCE LISTING (57 sequences)

SEQ ID NO: 1 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCDSIAFPSKTSASIGSLCADARMYGVLPWIAFPGKVCGSNLLSICKTAEFQMTFHLFIAAFVGAAATLVSLLTFMIAATYNFAVLKLMGRGTKF

SEQ ID NO: 2 - protein, 421 aa, Homo sapiens
MNRGFSRKSHTFLPKIFFRKMSSSGAKDKPELQFPFLQDEDTVATLLECKTLFILRGLPGSGKSTLARVIVDKYRDGTKMVSADAYKITPGARGAFSEEYKRLDEDLAAYCRRRDIRILVLDDTNHERERLEQLFEMADQYQYQVVLVEPKTAWRLDCAQLKEKNQWQLSADDLKKLKPGLEKDFLPLYFGWFLTKKSSETLRKAGQVFLEELGNHKAFKKELRQFVPGDEPREKMDLVTYFGKRPPGVLHCTTKFCDYGKAPGAEEYAQQDVLKKSYSKAFTLTISALFVTPKTTGARVELSEQQLQLWPSDVDKLSPTDNLPRGSRAHITLGCAADVEAVQTGLDLLEILRQEKGGSRGEEVGELSRGKLYSLGNGRWMLTLAKNMEVRAIFTGYYGKGKPVPTQGSRKGGALQSCTII

SEQ ID NO: 3 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 4 - protein, 582 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESKEVSTLESH

SEQ ID NO: 5 - DNA, 53 bp, Artificial Sequence
  Synthetic polynucleotide
gtcgtatccagtgcagggtccgaggtattcgcactggatacgacatgcttttc

SEQ ID NO: 6 - DNA, 20 bp, Artificial Sequence
  Synthetic polynucleotide
ggcctcgttctgagacctgg

SEQ ID NO: 7 - DNA, 16 bp, Artificial Sequence
  Synthetic polynucleotide
gtgcagggtccgaggt

SEQ ID NO: 8 - RNA, 91 nt, Artificial Sequence
  Synthetic polynucleotide
ucugcaccagaggcuucugagaccugggaaaagcauugugauugugauucagugcuuugcuugauccauguccagagucuucugcuucaga

SEQ ID NO: 9 - RNA, 22 nt, Artificial Sequence
  Synthetic polynucleotide
uucugagaccugggaaaagcau

SEQ ID NO: 10 - DNA, 22 bp, Artificial Sequence
  Synthetic polynucleotide
ttctgagacctgggaaaagcat

SEQ ID NO: 11 - RNA, 22 nt, Artificial Sequence
  Synthetic polynucleotide
ugcuugauccauguccagaguc

SEQ ID NO: 12 - DNA, 22 bp, Artificial Sequence
  Synthetic polynucleotide
tgcttgatccatgtccagagtc

SEQ ID NO: 13 - DNA, 20 bp, Artificial Sequence
  Synthetic polynucleotide
tgtgtgtctgcaccagaggc

SEQ ID NO: 14 - DNA, 19 bp, Artificial Sequence
  Synthetic polynucleotide
gtgtgtctgcaccagaggc

SEQ ID NO: 15 - DNA, 18 bp, Artificial Sequence
  Synthetic polynucleotide
tgtgtctgcaccagaggc

SEQ ID NO: 16 - DNA, 24 bp, Artificial Sequence
  Synthetic polynucleotide
cttctgagacctgggaaaagcatt

SEQ ID NO: 17 - DNA, 30 bp, Artificial Sequence
  Synthetic polynucleotide
ttctgagacctgggaaaagcattgtgattg

SEQ ID NO: 18 - DNA, 24 bp, Artificial Sequence
  Synthetic polynucleotide
ttctgagacctgggaaaagcattg

SEQ ID NO: 19 - DNA, 23 bp, Artificial Sequence
  Synthetic polynucleotide
ttctgagacctgggaaaagcatt

SEQ ID NO: 20 - DNA, 22 bp, Artificial Sequence
  Synthetic polynucleotide
ttctgagacctgggaaaagcat

SEQ ID NO: 21 - DNA, 21 bp, Artificial Sequence
  Synthetic polynucleotide
ttctgagacctgggaaaagca

SEQ ID NO: 22 - DNA, 20 bp, Artificial Sequence
  Synthetic polynucleotide
tgagacctgggaaaagcatt

SEQ ID NO: 23 - DNA, 22 bp, Artificial Sequence
  Synthetic polynucleotide
gtgcttgatccatgtccagagt

SEQ ID NO: 24 - DNA, 23 bp, Artificial Sequence
  Synthetic polynucleotide
tgcttgatccatgtccagagtct

SEQ ID NO: 25 - DNA, 22 bp, Artificial Sequence
  Synthetic polynucleotide
tgcttgatccatgtccagagtc

SEQ ID NO: 26 - DNA, 21 bp, Artificial Sequence
  Synthetic polynucleotide
ctgtgtgtctgcaccagaggc

SEQ ID NO: 27 - DNA, 20 bp, Artificial Sequence
  Synthetic polynucleotide
tgtgtgtctgcaccagaggc

SEQ ID NO: 28 - DNA, 17 bp, Artificial Sequence
  Synthetic polynucleotide
tgtgtgtctgcaccaga

SEQ ID NO: 29 - DNA, 19 bp, Artificial Sequence
  Synthetic polynucleotide
gtgtgtctgcaccagaggc

SEQ ID NO: 30 - DNA, 18 bp, Artificial Sequence
  Synthetic polynucleotide
tgtgtctgcaccagaggc

SEQ ID NO: 31 - DNA, 17 bp, Artificial Sequence
  Synthetic polynucleotide
gtgtctgcaccagaggc

SEQ ID NO: 32 - DNA, 30 bp, Artificial Sequence; identical to SEQ ID NO: 17
  Synthetic polynucleotide

SEQ ID NO: 33 - DNA, 25 bp, Artificial Sequence
  Synthetic polynucleotide
ttctgagacctgggaaaagcattgt

SEQ ID NO: 34 - DNA, 24 bp, Artificial Sequence
  Synthetic polynucleotide
ttctgagacctgggaaaagcattg

SEQ ID NO: 35 - DNA, 23 bp, Artificial Sequence
  Synthetic polynucleotide
ttctgagacctgggaaaagcatt

SEQ ID NO: 36 - DNA, 22 bp, Artificial Sequence
  Synthetic polynucleotide
ttctgagacctgggaaaagcat

SEQ ID NO: 37 - DNA, 21 bp, Artificial Sequence
  Synthetic polynucleotide
ttctgagacctgggaaaagca

SEQ ID NO: 38 - DNA, 20 bp, Artificial Sequence
  Synthetic polynucleotide
ttctgagacctgggaaaagc

SEQ ID NO: 39 - DNA, 18 bp, Artificial Sequence
  Synthetic polynucleotide
ttctgagacctgggaaaa

SEQ ID NO: 40 - DNA, 17 bp, Artificial Sequence
  Synthetic polynucleotide
ttctgagacctgggaaa

SEQ ID NO: 41 - DNA, 24 bp, Artificial Sequence
  Synthetic polynucleotide
tctgagacctgggaaaagcattgt

SEQ ID NO: 42 - DNA, 23 bp, Artificial Sequence
  Synthetic polynucleotide
tctgagacctgggaaaagcattg

SEQ ID NO: 43 - DNA, 22 bp, Artificial Sequence
  Synthetic polynucleotide
tctgagacctgggaaaagcatt

SEQ ID NO: 44 - DNA, 30 bp, Artificial Sequence
  Synthetic polynucleotide
tgtgattgtgattcagtgcttgatccatgt

SEQ ID NO: 45 - DNA, 30 bp, Artificial Sequence
  Synthetic polynucleotide
gtgattgtgattcagtgcttgatccatgtc

SEQ ID NO: 46 - DNA, 30 bp, Artificial Sequence
  Synthetic polynucleotide
attgtgattcagtgcttgatccatgtccag

SEQ ID NO: 47 - DNA, 30 bp, Artificial Sequence
  Synthetic polynucleotide
gtgattcagtgcttgatccatgtccagagt

SEQ ID NO: 48 - DNA, 30 bp, Artificial Sequence
  Synthetic polynucleotide
agtgcttgatccatgtccagagtcttctgc

SEQ ID NO: 49 - DNA, 22 bp, Artificial Sequence
  Synthetic polynucleotide
gtgcttgatccatgtccagagt

SEQ ID NO: 50 - DNA, 24 bp, Artificial Sequence
  Synthetic polynucleotide
tgcttgatccatgtccagagtctt

SEQ ID NO: 51 - DNA, 23 bp, Artificial Sequence
  Synthetic polynucleotide
tgcttgatccatgtccagagtct

SEQ ID NO: 52 - DNA, 22 bp, Artificial Sequence
  Synthetic polynucleotide
tgcttgatccatgtccagagtc

SEQ ID NO: 53 - DNA, 21 bp, Artificial Sequence
  Synthetic polynucleotide
tgcttgatccatgtccagagt

SEQ ID NO: 54 - DNA, 20 bp, Artificial Sequence
  Synthetic polynucleotide
tgcttgatccatgtccagag

SEQ ID NO: 55 - DNA, 19 bp, Artificial Sequence
  Synthetic polynucleotide
tgcttgatccatgtccaga

SEQ ID NO: 56 - DNA, 17 bp, Artificial Sequence
  Synthetic polynucleotide
tgcttgatccatgtcca

SEQ ID NO: 57 - DNA, 30 bp, Artificial Sequence
  Synthetic polynucleotide
ttctgcttcagaatcttcctctctaggaaa

(19) United States
(12) Patent Application Publication
(10) Pub. No.: US 2013/0273549 A1
(43) Pub. Date: Oct. 17, 2013

(54) DETECTION OF EXTRACELLULAR JCV MICRORNAS

(71) Applicant: BOARD OF REGENTS, THE UNIVERSITY OF TEXAS SYSTEM, Austin, TX (US)

(72) Inventors: Christopher Sullivan, Austin, TX (US); Chun Jun Chen, Austin, TX (US)

(21) Appl. No.: 13/864,188

(22) Filed: Apr. 16, 2013

Related U.S. Application Data
(60) Provisional application No. 61/624,956, filed on Apr. 16, 2012.

(51) Int. Cl. C12Q 1/68

(52) U.S. Cl. CPC C12Q 1/686
USPC 435/6.12; 536/24.1

(57) ABSTRACT

JCV-miRNA compositions and methods for detecting glia-derived JCV-miRNA are provided. The compositions and methods of the present invention are particularly useful as a non-invasive biomarker prognostic and/or diagnostic for patients suffering from PML.

### CROSS-REFERENCES TO RELATED APPLICATIONS

This application claims priority to U.S. Provisional Patent Application No. 61/624,956, filed Apr. 16, 2012, entitled “DETECTION OF EXTRACELLULAR JCV MICRORNAS” the disclosure of which is incorporated by reference herein in its entirety.

### REFERENCE TO A “SEQUENCE LISTING,” A TABLE, OR A COMPUTER PROGRAM LISTING APPENDIX SUBMITTED ON A COMPACT DISK

The Sequence Listing written in file 93331-872292_ST25.TXT, created on Apr. 16, 2013, 24,116 bytes, machine format IBM-PC, MS-Windows operating system, is hereby incorporated by reference.

### BACKGROUND OF THE INVENTION

Current diagnostics for Progressive Multifocal Leukoencephalopathy (PML) involve detection of JCV DNA via the polymerase chain reaction (PCR) from cerebrospinal fluid (CSF) or a direct brain biopsy (Brew et al., 2010; Major, 2010). Both of these assays are invasive and impractical for routine sampling. Other PML biomarker approaches have attempted to utilize PCR for JCV from isolated blood/serum or detection of immunoreactive antibodies against JCV. Both of these approaches fall short as biomarkers for PML since many healthy non-PML patients have been previously exposed to JCV and will periodically have JCV viremia. In fact, approximately 50 to 80% of the human population is seropositive for JCV antibodies as JCV is found to persistently infect the kidney and perhaps other non-neural tissues such as lymphocytes (Brew et al, 2010). Thus, being seropositive for JCV-reactive antibodies or even having viral DNA detected in bodily fluids via PCR is not predictive of the neural disease PML.

In patients with PML, magnetic resonance imaging (MRI) can sometimes be used to reveal changes in local brain features characteristic of this condition. However, this methodology misses many cases of PML and is not amenable as an early diagnostic of PML. Furthermore, other neurological disorders can cause white matter abnormalities such as multiple sclerosis and systemic lupus erythematosus (Brew et al., 2010). Therefore, at present, a definitive diagnosis for PML can only be confirmed by the detection of JCV DNA in the CSF or in brain biopsy, which is too invasive to be routinely performed as a prognostic diagnostic for rare incidences of PML associated with various drug regimens.

Several potentially important drugs that could benefit patients including: Natalizumab (trade name Tysabri) for multiple sclerosis, efalizumab (trade name Raptiva) for psoriasis and rituximab (trade name Rituxan) for arthritis are associated with rare occurrences of PML. The gold standard for the diagnosis of PML is through a brain biopsy, in combination with the detection of JCV DNA in the CSF via PCR. Both methodologies are invasive in nature. Thus, there is a need in the art for non-invasive and accurate methods for detecting JCV infections. The present invention addresses these and other needs in the art.

### BRIEF SUMMARY OF THE INVENTION

Disclosed herein, inter alia, are methods and materials useful to PML detection in AIDS patients and patients on autoimmune anti-inflammatory drugs. The methods and materials provided herein are of significant importance, for example, for a non-invasive early detection assay for PML. In some embodiments, the methods and compositions disclosed herein allow detection of JCV microRNAs as a biomarker for PML providing a non-invasive and sensitive prognostic/diagnostic tool that is currently lacking for PML. In some other embodiments, the methods and compositions disclosed herein proved detection of glia-derived exosomes having JCV-miRNA for use as a prognostic/diagnostic tool for non-invasive detection of PML.

In one aspect, an isolated glia-derived extracellular JCV-miRNA is provided. In some embodiments, the glia-derived extracellular JCV-miRNA is included in a JCV-miRNA infected exosome. In certain embodiments the isolated glia-derived extracellular JCV-miRNA is obtained from a mammal. In certain embodiments the isolated glia-derived extracellular JCV-miRNA is obtained from a human. In some embodiments, the human is suffering from PML. In certain embodiments the isolated glia-derived extracellular JCV-miRNA is derived from a fluid sample. In some embodiments, the fluid sample is a blood sample or a urine sample. In some embodiments, the blood sample is a blood plasma sample or a blood serum sample.

In another aspect, a JCV-miRNA infected glia-derived exosome is provided. In some embodiments, the JCV-miRNA infected glia-derived exosome is isolated from a mammalian subject. In certain embodiments, the JCV-miRNA infected glia-derived exosome is isolated from a human subject. In some embodiments, the subject is suffering from PML.

In another aspect, a method of detecting a glia-derived extracellular JCV miRNA in a sample derived from a subject is provided. The method includes isolating a glia-derived extracellular JCV miRNA within a sample derived from a subject thereby producing isolated glia-derived extracellular JCV miRNA. The isolated glia-derived extracellular JCV miRNA is reversed transcribed, thereby producing glia derived JCV cDNA. The glia-derived JCV cDNA is amplified thereby forming a plurality of amplified glia-derived JCV cDNAs and a plurality of complementary glia-derived JCV cDNAs. The presence of the plurality of amplified extracellular glia-derived JCV cDNAs or the plurality of complementary glia-derived JCV cDNAs is detected thereby detecting the glia-derived extracellular JCV miRNA in the sample.

### BRIEF DESCRIPTION OF THE DRAWINGS

FIG. 1: Exosomes isolated from JCV-infected glia cells. FIG. 1A and FIG. 1B: Exosomes were isolated from JCV Mad1 strain (one of the strains that cause PML) infected SVGA cells (astrocytes) by ultracentrifugation. FIGS. 1a and 1b show Transmission Electron Microscopy images taken from the exosomes preparation. Arrows indicate the exosomes isolated from the JCV infected SVGA culture supernatant. FIG. 1c shows a western blot analysis for CD63, a transmembrane protein that is enriched on exosomes.

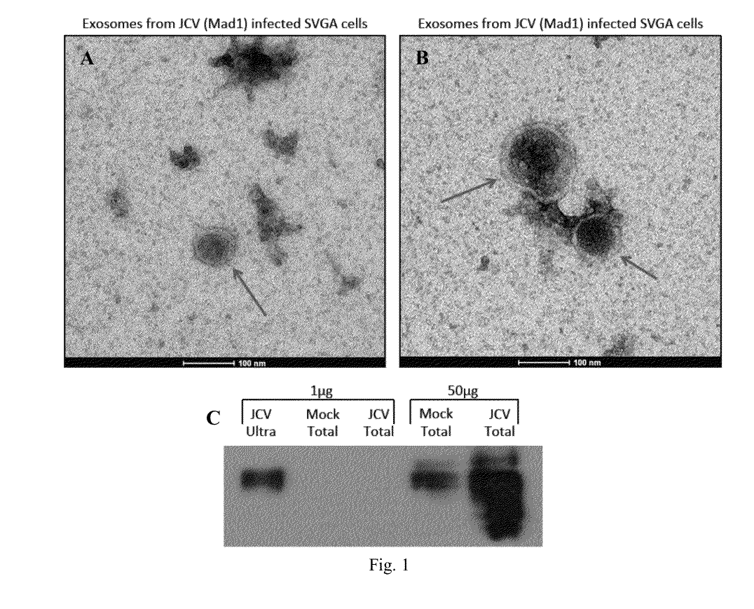

FIG. 2: RNA gel analysis shows JCV miRNA can be detected in exosomes isolated from JCV Mad1 infected SVGA cell culture supernatant. The box indicates that JCV miRNA is detectable in exosomes from JCV Mad1 infected SVGA cell culture supernatant but not from exosomes from uninfected SVGA cell culture supernatant. Lines - and 1-12 from left to right correspond to the following samples: −) negative control; 1) mock-infected total RNA without DNAse treatment at room temperature (RT); 2) mock-infected total RNA without DNAse treatment no RT; 3) mock-infected total RNA with DNAse treatment at RT; 4) mock-infected total RNA with DNAse treatment no RT; 5) JCV-infected total RNA without DNAse treatment at RT; 6) JCV-infected total RNA without DNAse treatment no RT; 7) JCV-infected total RNA with DNAse treatment at RT; 8) JCV-infected total RNA with DNAse treatment no RT; 9) mock-infected ultracentrifuged RNA at RT; 10) mock-infected ultracentrifuged RNA no RT; 11) JCV-infected ultracentrifuged RNA at RT; 12) JCV-infected ultracentrifuged RNA no RT;

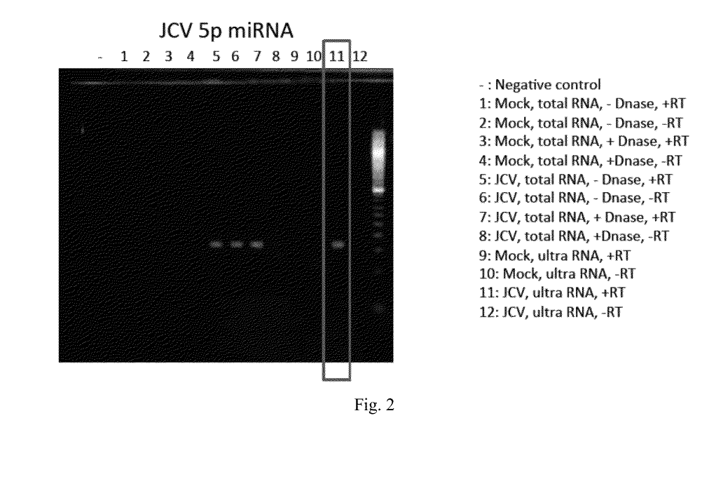

FIG. 3: Cartoon flow chart for a method of isolating JCV-miRNA from exosomes. Exosomes (1) that crosses the Blood-brain barrier (2) from a patient are isolated from either the blood serum (3) or from the urine samples (4). If exosomes of glial-origin is of high enough representation without enrichment in the blood serum or urine samples, quantitative stem-loop RT-PCR can be performed on the exosomes to detect JCV microRNAs. If enrichment is required, immunoaffinity pull-down (5) of glia exosomes can be performed and exosomes are captured (6) in microtiter plates for subsequent PCR analysis (7). After the enrichment process, quantitative stem-loop RT-PCR is performed to detect JCV microRNAs in those glia secreted exosomes.

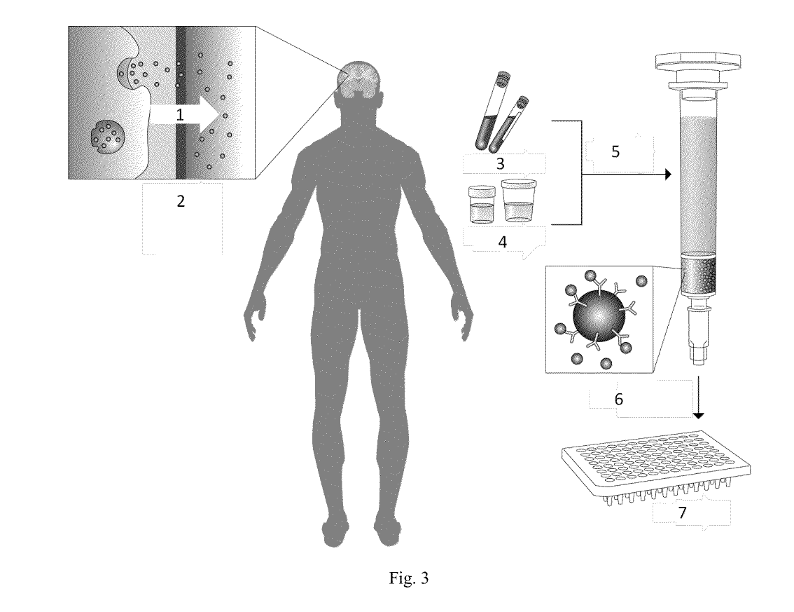

### DETAILED DESCRIPTION OF THE INVENTION

### I. Definitions

The following definitions are provided to facilitate understanding of certain terms used frequently herein and are not meant to limit the scope of the present disclosure. Unless defined otherwise, all technical and scientific terms used herein have the same meaning as commonly understood by one of ordinary skill in the art to which this invention belongs.

A “glia” or “glia cell” as used herein, refers to any neuroglial cell, such as an astrocyte or an oligodendrocyte. Glia cells do not participate directly in synaptic contacts. Instead, glia cells function to maintain the ionic milieu of nerve cells, modulate the rate of signal propagation, modulate uptake of neurotransmitters, provide scaffolding for neural development, and providing myelin sheets to some axons. There are three types of glia cells: microglia, astrocytes, and oligodendrocytes. Astrocytes regulate chemical environments whereas oligodendrocytes provide myelin sheets for some axons. (Purves D, Augustine G J, Fitzpatrick D, et al., editors. Neuroscience. 2nd edition. Sunderland (MA): Sinauer Associates; 2001. Neuroglial Cells.)

A “JCV-infected glia” as used herein refers to any glia cell having active JCV infection. In some embodiments, the glia actively secretes exosomes containing JCV-miRNA.

The term “nucleic acid monomers” refers to nucleoside or nucleotide. The nucleic acid monomers are typically useful precursors for making nucleic acid polymers (polynucleotides) enzymatically. Exemplary nucleic acid monomers include adenosine, guanosine, cytidine, uridine, thymidine, which may be present as monophosphates, diphosphates, and triphosphates. The term “polynucleotide” refers to a linear sequence of nucleotides joined by a phosphodiester linkage between the 5′ and 3′ carbon atoms.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but not to other sequences. Stringent conditions are sequence-dependent and will be different in different circumstances. Longer sequences hybridize specifically at higher temperatures. An extensive guide to the hybridization of nucleic acids is found in Tijssen, TECHNIQUES IN BIOCHEMISTRY AND MOLECULAR BIOLOGY—HYBRIDIZATION WITH NUCLEIC PROBES, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

The term “gene” refers to the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The term “amplifying” refers to a process in which the nucleic acid is exposed to at least one round of extension, replication, or transcription in order to increase (e.g., exponentially increase) the number of copies (including complementary copies) of the nucleic acid. The process can be iterative including multiple rounds of extension, replication, or transcription. Various nucleic acid amplification techniques are known in the art, such as PCR amplification, primer extension qPCR, rolling circle miRNA amplification, and RT-PCR. RT-PCR amplification of miRNA may be done using stem-loop RT-PCR techniques as described by Chen, et al, 2005. As used herein, “reverse transcribing” a miRNA to cDNA is done by performing RNA transcription to form cDNA (e.g. via RT-PCR). The reverse transcribing may be performed using available miRNA through the action of a reverse transcriptase, such as SuperScript®III (Invitrogen).

A “primer”, as used herein, refers to a nucleic acid that is capable of hybridizing to a complementary nucleic acid sequence in order to facilitate enzymatic extension, replication or transcription. The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. Non-limiting examples of primers useful for the detection of glia-derived JCV-cDNA are set forth in Table 3. In some embodiments, the glia-derived JCV-cDNA is amplified using the primers set forth in Table 3.

An “PLP protein” as referred to herein includes any of the naturally-occurring forms of the myelin proteolipid protein, or variants thereof that maintain PLP protein activity (e.g. within at least 50%, 80%, 90%, 95%, 96%, 97%, 98%, 99% or 100% activity compared to PLP). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PLP polypeptide (e.g. SEQ ID NO:1). In other embodiments, the PLP protein is the protein as identified by the NCBI reference gi:187417 corresponding to SEQ ID NO:1.

An “CNP protein” as referred to herein includes any of the naturally-occurring forms of the 2′,3′-cyclic-nucleotide 3′-phosphodiesterase protein, or variants thereof that maintain CNP protein activity (e.g. within at least 50%, 80%, 90%, 95%, 96%, 97%, 98%, 99% or 100% activity compared to CNP). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CNP polypeptide (e.g. SEQ ID NO:2). In other embodiments, the CNP protein is the protein as identified by the NCBI reference gi:94721261 corresponding to SEQ ID NO:2.

An “MOG protein” as referred to herein includes any of the naturally-occurring forms of the myelin oligodendrocyte glycoprotein, or variants thereof that maintain MOG protein activity (e.g. within at least 50%, 80%, 90%, 95%, 96%, 97%, 98%, 99% or 100% activity compared to MOG. In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MOG polypeptide (e.g. SEQ ID NO:3). In other embodiments, the MOG protein is the protein as identified by the NCBI reference gi:984147 corresponding to SEQ ID NO:3.

An “MAG protein” as referred to herein includes any of the naturally-occurring forms of the myelin-associated glycoprotein, or variants thereof that maintain MAG protein activity (e.g. within at least 50%, 80%, 90%, 95%, 96%, 97%, 98%, 99% or 100% activity compared to MAG. In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MAG polypeptide (e.g. SEQ ID NO:4). In other embodiments, the MAG protein is the protein as identified by the NCBI reference gi:62205282 corresponding to SEQ ID NO:4.

A “JCV-miRNA nucleic acid probe”, as used herein, is a nucleic acid fragment used in combination with a RT and nucleic acid monomers for amplification and detection of JCV-miRNA. The JCV-miRNA nucleic acid probe is typically of sufficient length and complementarity to a JCV genomic sequence as to adequately serve as a primer for amplification. In some embodiments, the complementarity of the JCV-miRNA nucleic acid probe to a JCV genomic sequence is at least about 60%, preferably about 65%, 70%, 75%, 80%, 85%, 90%, 91%, 92%, 93%, 94%, 95%, 96%, 97%, 98%, 99%, or higher.

The term “sample” includes sections of tissues such as biopsy and autopsy samples, and frozen sections taken for histological purposes. Such samples include blood and blood fractions or products (e.g., serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish. In some embodiments, the sample is obtained from a human.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. Examples of control samples include but are not limited to; a control sample level from a healthy patient sample(s), a level taken from a known PML patient sample(s), a level from a patient with known JCV viremia, a level from a sample from the same subject taken at an earlier time, or any combination thereof.

The term “isolated” refers to any cell or cellular component such as a nucleotide, polynucleotide, peptide, polypeptide, or exosome, that is partially or completely separated from components with which it is naturally associated (such as proteins, nucleic acids, cells, tissues).

As used herein, “PML” or “progressive multifocal leukoencephalopathy” refers to a fatal neurological condition consistently associated with JC virus infection. PML can occur in any white matter regions of the brain resulting in demyelination of neurons. Atypical oligodendrocytes and extremely large astrocytes are found in the brains of PML patients. Previously a rare condition, generally occurs in patients with marked cellular immunodeficiency. Thus, PML has become more prevalent with the emergence of HIV. PML is also associated with transplantation rejection, leukemia patients, patients with chronic inflammation and has recently been associated with immunosuppressant antibody drugs such as Natalizumab. Detection of PML may be performed through brain biopsy, PCR detection of JCV DNA in the CSF, MRI detection of focal abnormalities, or through observation of worsening neurological symptoms.

“JC virus” or “John Cunningham Virus” or “JCV” is a member of the Polyomavirus family. JCV typically has a small circular DNA genome of approximately 5 kilobases. JCV infection is associated with PML. JCV infection, either latent or active, is common and it is estimated between 50-80% of the population is seropositive for JCV antibodies. JCV can cross the blood brain barrier and upon reactivation, typically due to immunodeficiency or immunosuppression, causes focal demyelination leading to PML.

As used herein, the term “miRNA” or “microRNA” is used herein according to its normal meaning as single-stranded RNA molecules of about 17-25 (e.g. 17-23) nucleotides in length typically capable of modifying gene expression and primary transcripts (pri-miRNAs), pre-cursors such as stem-loop precursors (pre-miRNAs) and variants thereof. Naturally-occurring miRNAs are typically transcribed from genes but are not translated into protein. miRNA's are typically first transcribed as a pri-miRNA (from about 45 nt's to 1000's of nts) that may include at least one hairpin structure (from about 37-120 nt). The pri-miRNA is typically processed to short pre-miRNA stem-loop precursors of about 40-80 nt. The sequence of the pre-miRNA may include the entire miRNA sequence. The sequence of the pre-miRNA may comprise the sequence of a hairpin loop. Pre-miRNA is typically processed by cleavage of a stem-loop, forming mature miRNAs of about 17-25 nt derived from either or both arms of the hairpin.

miRNA sequences are involved in various physiological and pathological conditions, including differentiation, development, tumorigenesis, and neurological disorders. miRNAs may function by binding to the 3′UTR of target mRNAs and inhibiting expression of protein from these transcripts. miRNAs may also direct cleavage of transcripts if they have perfect complementarity to such transcript.

As used herein, the term “viral miRNA” or “viral microRNA” refers to miRNA having a viral origin. Viral miRNA may be encoded by viruses with DNA genomes or retroviruses with RNA genomes. Viral miRNA may cleave early viral mRNA transcripts or host gene transcripts.

“JCV-miRNA” refers to a viral miRNA encoded by JC virus. In one such example, JCV encodes an approximately 60 nt pre-miRNA that is processed into different mature miRNAs approximately 22 nt in length.

As used herein, the term “glia-derived extracellular JCV-miRNA” refers to a miRNA derived from a JCV infected glial cell. Thus, the glia-derived extracellar JCV-miRNA typically has a JC viral origin in which the JCV has infected a glial cell (such as an oligodendrocyte or astrocyte). The glia-derived extracellular JCV-miRNA is typically present outside of the cellular matrix of the glial cell. An “isolated glia-derived extracellular JCV-miRNA” is a glia-derived extracellular JCV-miRNA that is partially or completely separated from components found in the sample in which the glia-derived extracellular JCV-miRNA is found. In some embodiments, the glia-derived extracellular JCV-miRNA includes a nucleotide sequence corresponding to SEQ ID NO:8 or a functional fragment thereof. In some embodiments, the glia-derived extracellular JCV-miRNA includes a nucleotide sequence corresponding to SEQ ID NO:9 or a functional fragment thereof. In some embodiments, the glia-derived extracellular JCV-miRNA includes a nucleotide sequence corresponding to SEQ ID NO:11 or a functional fragment thereof. In some embodiments, the glia-derived extracellular JCV-miRNA has the nucleotide sequence of SEQ ID NO:8. In some embodiments, the glia-derived extracellular JCV-miRNA has the nucleotide sequence of SEQ ID NO:9. In some embodiments, the glia-derived extracellular JCV-miRNA has the nucleotide sequence of SEQ ID NO:11.

An “enriched glia-derived extracellular JCV-miRNA sample fraction” is a collection of glia-derived extracellular JCV-miRNAs derived from a sample having glia-derived extracellular JCV-miRNAs, in which the concentration of the glia-derived extracellular JCV-miRNAs is increased relative to the concentration of the glia-derived extracellular JCV-miRNAs in the sample from which the sample fraction is derived. A variety of enrichment techniques may be used, such as differential centrifugation or column chromatography including size exclusion chromatography or affinity chromatography.

The term “glia-derived JCV-cDNAs” refers to a cDNA complementary to glia-derived JCV-miRNA (prepared e.g. via RT-PCR). In some embodiment, the glia-derived JCV-cDNA matches the original DNA sequence encoding the JCV-miRNA. “Complementary glia-derived JCV-cDNA” refers to synthesized strands of cDNA complementary to glia-derived JCV-cDNA. In some embodiments, the complementary glia-derived JCV-cDNA matches the original miRNA sequence.

As used herein, the term “exosome” refers to a microvesicle secreted into the extracellular milieu from a cell, such as a glial cell (e.g. oligodendrocytes or astrocytes). Exosome secretion typically occurs through reverse budding of the limiting membrane of multivesicular endosomes forming microvesicles (e.g. of about 50-100 nm in diameter). Exosomes may have the capability of crossing the blood-brain barrier. Exosomes may contain cytosol and extracellular domains of membrane-bound cellular proteins on their surface. As such, exosomes may also contain additional cellular components such as nucleic acids, peptides, and proteins. Exosomes may also contain miRNA from either or both the host or a virus. Exosomes may be released at higher rates during infection with certain viruses or other various stressors. The release of exosomes may increase intercellular communication. Exosomes may be extracted from biological fluids such as blood, serum, urine. As used herein, a “glia-derived exosome” refers to an exosome originating from a glial cell.

A “JCV-miRNA infected glia exosome”, as used herein, refers to an exosome derived from a glial cell, such as an astrocyte or oligodendrocyte, which has at least one JCV-miRNA contained therein.

A “glia-derived exosome binding protein”, as used herein, refers to protein (e.g. peptide or polypeptide) that binds to an exosome (e.g. to a protein found on the surface of an exosome in nature, commonly referred to as an exosomal surface protein). Examples of exosomal surface proteins useful for the invention provided herein including embodiments thereof are without limitation major myelin proteolipid protein (PLP), 2′3′-cyclic-nucleotide-phosphodiesterase (CNP), myelin oligodendrocyte glycoprotein (MOG) or myelin-associated glycoprotein (MAG). Glia-derived exosome binding proteins can be conjugated with exogenously added conjugates for detection or purification chromatographic techniques such as affinity chromatography or antibody-based immunoprecipitation. An “exosome-binding protein complex”, as used herein, refers to a glia-derived exosome binding protein covalently or non-covalently bound to an exosome (e.g. to an exosomal surface protein). In some embodiments, complexing occurs when a glia-derived exosome binding protein binds to an exosomal surface protein. Such complexes may be formed in vitro and are useful for detection and purification. An “exosome binding antibody”, as used herein, refers to an antibody capable of recognizing glia-derived exosomes. Exosome binding antibodies may be used for affinity chromatography or immunoprecipitation of glia-derived exosomes.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

### II. Compositions

In one aspect, an isolated glia-derived extracellular JCV-miRNA is provided. In some embodiments, the glia-derived extracellular JCV-miRNA is included in a JCV-miRNA infected exosome. In certain embodiments the isolated glia-derived extracellular JCV-miRNA is obtained from a mammal. In certain embodiments the isolated glia-derived extracellular JCV-miRNA is obtained from a human. In some embodiments, the human is suffering from PML. In certain embodiments the isolated glia-derived extracellular JCV-miRNA is derived from a fluid sample. In some embodiments, the fluid sample is a blood sample or a urine sample. In some embodiments, the blood sample is a blood plasma sample or a blood serum sample.

In another aspect, a JCV-miRNA infected glia-derived exosome is provided. In some embodiments, the JCV-miRNA infected glia-derived exosome is isolated from a mammalian subject. In certain embodiments, the JCV-miRNA infected glia-derived exosome is isolated from a human subject. In some embodiments, the subject is suffering from PML.

### III. Methods of Detection

In another aspect, method of detecting a glia-derived extracellular JCV miRNA in a sample derived from a subject is provided. The method includes isolating a glia-derived extracellular JCV miRNA within a sample derived from a subject thereby producing isolated glia-derived extracellular JCV miRNA. The isolated glia-derived extracellular JCV miRNA is reversed transcribed, thereby producing glia derived JCV cDNA. The glia-derived JCV cDNA is amplified thereby forming a plurality of amplified glia-derived JCV cDNAs and a plurality of complementary glia-derived JCV cDNAs. The presence of the plurality of amplified extracellular glia-derived JCV cDNAs or the plurality of complementary glia-derived JCV cDNAs is detected thereby detecting the glia-derived extracellular JCV miRNA in the sample. In embodiments, the sample is obtained from the subject.

In some embodiments, the detecting the glia-derived extracellular JCV-miRNA in the sample indicates detecting a JCV-infected glia within the subject. In other embodiments, the detecting includes determining an amount of the plurality of amplified glia-derived extracellular JCV-cDNAs or the plurality of complementary glia-derived extracellular JCV-cDNAs. Based on the amount, a level of glia-derived extracellular JCV-miRNAs is determined within the sample. And the level is compared to a standard control level, wherein the level being higher than the standard control level is indicative of the subject having PML.

In some embodiments, the subject is a mammalian subject. In other embodiments, the subject is a human subject. In some embodiments, the subject is a PML patient.

In some embodiments, the sample is a fluid sample. In other embodiments, the sample is a blood sample or a urine sample. In some embodiments, the blood sample is a blood plasma sample. In other embodiments the blood sample is a blood serum sample.

In some embodiments, the isolating includes centrifuging the sample under conditions suitable to form an enriched glia-derived extracellular JCV-miRNA sample fraction. In some embodiments, the glia-derived extracellular JCV-miRNA within the sample forms part of a JCV-miRNA infected glia-derived exosome. In other embodiments, the isolating includes separating the JCV-miRNA infected glia-derived exosome from components of the sample. In some embodiments, the separating includes contacting the JCV-miRNA infected glia-derived exosome with a glia-derived exosome-binding protein to form an glia-derived exosome-binding protein complex and isolating the exosome-binding protein complex. In some embodiments, the glia-derived exosome-binding protein is a glia-derived exosome binding antibody. In some embodiments, the glia-derived exosome binding antibody is capable of binding a myelin proteolipid (PLP) protein. In other embodiments, the glia-derived exosome binding antibody is capable of binding a 2′,3′-cyclic-nucleotide 3′-phosphodiesterase (CNP). In other embodiments, the glia-derived exosome binding antibody is capable of binding a myelin oligodendrocyte glycoprotein (MOG). In other embodiments, the glia-derived exosome binding antibody is capable of binding a myelin-associated glycoprotein (MAG).

In some embodiments, the reverse transcribing includes contacting the isolated glia-derived extracellular JCV-miRNA with a JCV-miRNA nucleic acid probe, a reverse transcriptase and nucleic acid monomers.

### IV. Examples

Cell Culture and Preparation of Culture Medium for Exosomes Isolation

SVGA cells (SV40 transformed astroglial cells, kindly provided by Walter Atwood, Brown University, Providence, R.I.) were maintained in Minimal Essential Medium Eagle with Earle's salts and L-glutamine (MEM, Cellgro, Manassa, Va.) supplemented with 10% heat inactivated fetal bovine serum (FBS, Sigma-Aldrich, St. Louis, Mo.), penicillin (100 IU/mL) and streptomycin (100 μg/mL) (Cellgro).

SVGA cells were infected with JCV (Mad1 strain, kindly provided by Walter Atwood) in MEM containing 2% heat inactivated FBS at 37° C. for 1 hour. The virus inoculum was replaced with 30 mL of regular MEM containing 10% heat inactivated FBS.

Ultracentrifugation Isolation of Exosomes

Exosomes were isolated from either mock-infected or JCV-infected SVGA cell culture media by first centrifugation at 300 g for 10 minutes at 4° C. (Avanti J-E centrifuge with JS 5.3 swinging bucket rotor, Beckman Coulter, Brea, Calif.). The supernatant was collected and centrifuge at 2000×g for 20 minutes at 4° C. (Avanti J-E centrifuge with a JS 5.3 swinging bucket rotor, Beckman Coulter). The supernatant was then transferred to a 75 mL polycarbonate bottles (ThermoFisher Scientific, Asheville, N.C.). The supernatant was centrifuged at 10,000×g for 30 minutes at 4° C. (Sorvall Ultra Pro 80 with a T-647.5 fixed angle rotor, ThermoFisher Scientific). The supernatant is transferred using a pipet to a fresh 75 mL polycarbonate bottle and centrifuged at 100,000 g for 2 hours and 30 minutes at 4° C. The supernatant was discarded by decanting. The pellet was washed with phosphate-buffered saline (PBS, Cellgro) and centrifuged at 100,000 g for 2 hours and 30 minutes at 4° C. The supernatant was then discarded by decanting. To remove the supernatant as completely as possible, the bottle was kept upside down and the remaining liquid on the side of the mouth of the bottle was removed with an aspirator. The pellet is suspended in 200 μL of PBS and divided into 50 μL aliquots and stored at −80° C.

RNA Isolation and Stem-Loop RT PCR Detection of JCV microRNA

Total RNA from mock or JCV-infected SVGA cells were harvested using an in-house PIG-B solution (2M guanidinium thiocyanate (EMD, Billerica, Mass.), 20 mM citrate buffer, pH4.5, 5 mM EDTA (Fisher Scientific, Pittsburgh, Pa.), 0.25% Sarkosyl (Sigma Aldrich), 48% saturated phenol, pH4.5 (Amresco, Solon, Ohio), 2.1% isoamyl alcohol (Fisher Scientific), 0.5% β-mercaptoethanol (Sigma Aldrich), 0.1% 8-hydroxyquinoline (EMD), and 0.0025% Coomassie blue (EMD)) as described previously (1, 2, 3). RNA from mock-infected or JCV-infected SVGA exosomes were isolated using the same protocol as above.

Prior to reverse transcription, total RNA was either left untreated or treated with TURBO DNase (Ambion, Austin, Tex.) according to the manufacturer's protocol. Reverse transcription was performed using SuperScript III reverse transcriptase (Invitrogen, Grand Island, N.Y.) according to the manufacturer's protocols. PCR was conducted on the reverse transcription product using Taq DNA polymerase (New England BioLabs, Ipswich, Mass.). The primers used were as follows:

JCV 5p miRNA stem-loop RT primer:
GTCGTATCCAGTGCAGGGTCCGAGGTATTCGCACTGGATACGACATGCT
TTTC,
JCV 5p miRNA PCR forward primer:
GGCCTCGTTCTGAGACCTGG,
Stem-loop PCR reverse primer:
GTGCAGGGTCCGAGGT.

The PCR program used was a touchdown PCR protocol with the annealing temperature starting at 62° C. for 30 seconds, decreasing by 0.5° C. each cycle for 20 cycles.

PCR was continued for another 25 cycles at an annealing temperature of 52° C. The extension step was performed at 68° C. for 30 seconds for each cycle. The PCR product was analyzed in a 3% agarose Lithium Borate gel (BioExpress, Kaysville, Utah). The agarose gel was stained by ethidium bromide (Fisher Scientific) and visualized using a Bio-Rad Universal Hood II gel imager (Bio-Rad, Hercules, Calif.).

Protein Isolation and Western Blot Analysis

Mock or JCV-infected SVGA cells were lysed in RIPA buffer (150 mM sodium chloride (Avantor Performance Materials, Center Valley, Pa.), 10 mM Tris, pH 7.2 (Fisher Scientific), 0.1% sodium dodecyl sulfate (Avantor Performance Materials), 1% Triton X-100 (Fisher Scientific), 1% sodium deoxycholate (Alfa Aesar, Ward Hill, Mass.), 5 mM EDTA (Fisher Scientific) and 1 tablet of complete mini, EDTA-free protease inhibitor (Roche, Indianapolis, Ind.) per 10 mL of RIPA buffer.

1 μg of exosomes samples and 50 μg of mock or JCV-infected SVGA total protein were heated at 70° C. for 10 minutes in SDS sample buffer without β-mercaptoethanol (375 mM Tris-HCL, pH6.8 (Fisher Scientific), 6% (w/v) sodium dodecyl sulfate (Avantor Performance Materials), 48% (v/v) glycerol (Sigma-Aldrich), and 0.03% (w/v) bromophenol blue (Fisher Scientific) and subjected to electrophoresis using a 10% denaturing acrylamide gel (Bio-Rad). Proteins were transferred onto Immobilon-FL PVDF membrane (Millipore, Billerica, Mass.) using the Bio-Rad Mini Trans-Blot electrophoretic transfer cell (Bio-Rad). The membrane was blocked in 5% (w/v) skim milk powder (HEB, San Antonio, Tex.) in Tris-buffered saline with 0.1% (v/v) Tween-20 (TBST, Fisher Scientific) for 1 hour at room temperature. The membrane was probed for CD63 using a mouse monoclonal CD63 antibody (1:1000, Santa Cruz Biotechnology, Santa Cruz, Calif.), overnight at 4° C. After overnight incubation, the membrane was washed with TBST, 4 times, 15 minutes each, followed by incubation with anti-mouse secondary antibody IgG conjugated to HRP (1:5000, Invitrogen) for 1 hour at room temperature. The membrane was washed 4 times with TBST, 15 minutes each. The West Dura chemiluminescent substrate (Thermo Scientific) was used to generate the light signal for visualization on the Blue Ultra Autorad film (BioExpress).

Transmission Electron Microscopy (TEM)

Transmission electron microscopy imaging of exosome preparations was performed as described (4), with slight modifications. Briefly, 10 μL of the exosome preparation was mixed with 10 μL of 4% paraformaldehyde (PFA) solution in PBS (USB Corporation, Cleveland, Ohio) to achieve a final concentration of 2% PFA. A drop of the sample was spotted on a piece of parafilm (Pechiney Plastic Packaging, Menasha, Wis.) and a Formvar Carbon Film on 300 square mesh Nickel Grid (Electron Microscopy Sciences, Hatfield, Pa.) was floated on top of the sample. The excess liquid was blotted off and allowed to dry for 20 minutes at room temperature. The grids were washed 8 times with deionized water prior to staining, using the floating method as described above. To stain the samples, a drop of uranyl-acetate solution, pH4.2-4.5 (kindly provided by the Institute of Cellular and Molecular Biology Microscopy Facility, The University of Texas at Austin, Austin, Tex.) was spotted onto a piece of parafilm and the grid was floated on the drop for 5 minutes, followed by blotting off the excess uranyl-acetate solution. The grids were washed one time as described above. Imaging was performed using a Tecnai Spirit BioTwin transmission electron microscope (FEI, Hillsboro, Oreg.) at 80 kV.

Pre-Enrichment of Exosomes from Cell Culture Media

Prior to isolation of the neuronal exosomes via proteolipid protein (PLP) specific immunoaffinity capture, supernatant from SVGA cell cultures (mock or infected with JCV Mad1 strain) were subjected to ExoQuick-TC Exosome Precipitation (System Biosciences, Mountain View, Calif.), according to the manufacturer's protocol. Briefly, SVGA cell culture supernatant was centrifuged at 3000 g for 15 minutes at 4° C. 10 mL of the centrifuged SVGA cell culture supernatant was then mixed with 2 mL of ExoQuick-TC Exosome Precipitation Solution. The mixture was placed at 4° C. for overnight. After overnight incubation, the mixture was centrifuged at 1500 g for 30 minutes at 4° C. The resulting supernatant was aspirated. Another centrifugation step of 1500 g at 4° C. was done for an additional 5 minutes. The remaining trace of supernatant was aspirated. The exosome pellet was dissolved in 200 μL of PBS and stored at −80° C. As a positive control, anti-CD63 antibody which recognizes both neuronal and non-neuronal exosomes was used.

Immunoaffinity Capture of Exosomes from ExoQuick-TC Enriched Exosomes

200 μL of either EcoMag™ Protein L Magnetic Beads (Bioclone Inc., San Diego, Calif.) or Dynabeads® Protein G beads (Invitrogen, Grand Island, N.Y.) were washed twice with PBS+0.1% Tween 20 (ThermoFisher Scientific, Asheville, N.C.). 10 mg of the following PLP antibodies were added to 200 μL of the beads in a total volume of 400 μL of PBS+0.1% Tween 20 for conjugations to the beads, overnight, at 4° C., with rotation on a Labquake Shaker Rotisserie (ThermoFisher Scientific).

TABLE 1
Monoclonal antibodies (glia-derived exosome-binding antibodies) useful
for isolation of JCV-miRNA infected glia-derived exosomes.

| Antibody | Antigen |
|---|---|
| Monoclonal Mouse IgM Clone#O10 Human PLP Antibody | PLP |
| (R&D Systems, Inc., Minneapolis, MN) | protein |
| Monoclonal Mouse IgG2a kappa Myelin PLP Antibody (2D7) | PLP |
| (Novus Biologicals, Littleton, CO) | protein |
| Polyclonal Goat PLP Antibody (G-17) (Santa Cruz Biotech- | PLP |
| nology, Inc., Dallas, TX) | protein |
| Monoclonal Rat Myelin PLP (Human) Antibody (ImmunoDiag- | PLP |
| nostics, Inc., Woburn, MA) | protein |

After overnight antibody conjugation onto the magnetic beads, the beads were washed twice with PBS+0.1% BSA (Sigma-Aldrich, St. Louis, Mo.). 50 μL of the ExoQuick-TC enriched exosomes and 50 μL of PBS+0.1% BSA were added to the antibody-conjugated magnetic beads. Immunoaffinity capturing of the neuronal exosomes was done overnight, at 4° C., with rotation as above. After overnight capturing of the neuronal exosomes, the magnetic beads were washed three times with PBS+0.1% BSA. The exosomes-captured magnetic beads are subjected to RNA isolation for stem-loop RT PCR detection of JCV microRNA or western blot analysis.

TABLE 2
Glia-derived extracellular JCV-miRNA.

| SEQ ID NO: | microRNA | Sequences (RNA) | Sequences (DNA) | Accession# |
|---|---|---|---|---|
| SEQ ID NO: 8 | jcv-mir-J1 | ucugcaccagaggcuucugagaccug | | MI0009980 |
| | | ggaaaagcauugugauugugauuca | | |
| | | gugcuuugcuugauccauguccaga | | |
| | | gucu ucugcuucaga | | |
| SEQ ID NO: 9 | jcv-mir-J1-5p | uucugagaccugggaaaagcau | | MIMAT0009147 |
| SEQ ID NO: 10 | jcv-mir-J1-3p | ugcuugauccauguccagaguc | | MIMAT0009148 |
| SEQ ID NO: 11 | jcv-mir-J1-5p | | TTCTGAGACCTGGGAAAA | |
| | | | GCAT | |
| SEQ ID NO: 12 | jcv-mir-J1-3p | | TGCTTGATCCATGTCCAG | |
| | | | AGTC | |

TABLE 3
Primer Pairs for Amplification of glia-derived extracellular JCV-cDNAs.

| SEQ ID NO: | microRNA | Sequences (RNA) | Length | Counts |
|---|---|---|---|---|
| SEQ ID NO: 13 | 5′ miRNA | TGTGTGTCTGCACCAGAGGC | 20 | 5 |
| SEQ ID NO: 14 | | GTGTGTCTGCACCAGAGGC | 19 | 3 |
| SEQ ID NO: 15 | | TGTGTCTGCACCAGAGGC | 18 | 1 |
| SEQ ID NO: 16 | 5p miRNA | CTTCTGAGACCTGGGAAAAGCATT | 24 | 1 |
| SEQ ID NO: 17 | | TTCTGAGACCTGGGAAAAGCATTGTGATTG | 30 | 1 |
| SEQ ID NO: 18 | | TTCTGAGACCTGGGAAAAGCATTG | 24 | 4 |
| SEQ ID NO: 19 | | TTCTGAGACCTGGGAAAAGCATT | 23 | 7 |
| SEQ ID NO: 20 | | TTCTGAGACCTGGGAAAAGCAT | 22 | 2 |
| SEQ ID NO: 21 | | TTCTGAGACCTGGGAAAAGCA | 21 | 1 |
| SEQ ID NO: 22 | | TGAGACCTGGGAAAAGCATT | 20 | 1 |
| SEQ ID NO: 23 | 3p miRNA | GTGCTTGATCCATGTCCAGAGT | 22 | 6 |
| SEQ ID NO: 24 | | TGCTTGATCCATGTCCAGAGTCT | 23 | 1 |
| SEQ ID NO: 25 | | TGCTTGATCCATGTCCAGAGTC | 22 | 3 |
| SEQ ID NO: 26 | 5′ miRNA | CTGTGTGTCTGCACCAGAGGC | 21 | 1 |
| SEQ ID NO: 27 | | TGTGTGTCTGCACCAGAGGC | 20 | 14 |
| SEQ ID NO: 28 | | TGTGTGTCTGCACCAGA | 17 | 1 |
| SEQ ID NO: 29 | | GTGTGTCTGCACCAGAGGC | 19 | 16 |
| SEQ ID NO: 30 | | TGTGTCTGCACCAGAGGC | 18 | 2 |
| SEQ ID NO: 31 | | GTGTCTGCACCAGAGGC | 17 | 6 |
| SEQ ID NO: 32 | 5p miRNA | TTCTGAGACCTGGGAAAAGCATTGTGATTG | 30 | 5 |
| SEQ ID NO: 33 | | TTCTGAGACCTGGGAAAAGCATTGT | 25 | 2 |
| SEQ ID NO: 34 | | TTCTGAGACCTGGGAAAAGCATTG | 24 | 4 |
| SEQ ID NO: 35 | | TTCTGAGACCTGGGAAAAGCATT | 23 | 10 |
| SEQ ID NO: 36 | | TTCTGAGACCTGGGAAAAGCAT | 22 | 3 |
| SEQ ID NO: 37 | | TTCTGAGACCTGGGAAAAGCA | 21 | 3 |
| SEQ ID NO: 38 | | TTCTGAGACCTGGGAAAAGC | 20 | 1 |
| SEQ ID NO: 39 | | TTCTGAGACCTGGGAAAA | 18 | 2 |
| SEQ ID NO: 40 | | TTCTGAGACCTGGGAAA | 17 | 1 |
| SEQ ID NO: 41 | | TCTGAGACCTGGGAAAAGCATTGT | 24 | 1 |
| SEQ ID NO: 42 | | TCTGAGACCTGGGAAAAGCATTG | 23 | 1 |
| SEQ ID NO: 43 | | TCTGAGACCTGGGAAAAGCATT | 22 | 1 |
| SEQ ID NO: 44 | 3p miRNA | TGTGATTGTGATTCAGTGCTTGATCCATGT | 30 | 2 |
| SEQ ID NO: 45 | | GTGATTGTGATTCAGTGCTTGATCCATGTC | 30 | 25 |
| SEQ ID NO: 46 | | ATTGTGATTCAGTGCTTGATCCATGTCCAG | 30 | 3 |
| SEQ ID NO: 47 | | GTGATTCAGTGCTTGATCCATGTCCAGAGT | 30 | 1 |
| SEQ ID NO: 48 | | AGTGCTTGATCCATGTCCAGAGTCTTCTGC | 30 | 1 |
| SEQ ID NO: 49 | | GTGCTTGATCCATGTCCAGAGT | 22 | 1 |
| SEQ ID NO: 50 | | TGCTTGATCCATGTCCAGAGTCTT | 24 | 1 |
| SEQ ID NO: 51 | | TGCTTGATCCATGTCCAGAGTCT | 23 | 11 |
| SEQ ID NO: 52 | | TGCTTGATCCATGTCCAGAGTC | 22 | 42 |
| SEQ ID NO: 53 | | TGCTTGATCCATGTCCAGAGT | 21 | 25 |
| SEQ ID NO: 54 | | TGCTTGATCCATGTCCAGAG | 20 | 1 |
| SEQ ID NO: 55 | | TGCTTGATCCATGTCCAGA | 19 | 1 |
| SEQ ID NO: 56 | | TGCTTGATCCATGTCCA | 17 | 1 |
| SEQ ID NO: 57 | 3′ miRNA | TTCTGCTTCAGAATCTTCCTCTCTAGGAAA | 30 | 15 |

### V. References

Brew, B. J., et al. 2010. Progressive multifocal leukoencephalopathy and other forms of JC virus disease. Nat. Rev. Neurol. Dec; 6(12): 667-679.
Major, E. O. Progressive multifocal leukoencephalopathy in patients on immunomodulatory therapies. Annu Rev. Med. 2010; 61: 35-47.
Lin, Y. T., et al. 2010. Small RNA profiling reveals antisense transcription throughout the KSHV genome and novel small RNAs. RNA 16: 1540-1558.
Seo, G. J., L. H. Fink, B. O'Hara, W. J. Atwood, and C. S. Sullivan. 2008. Evolutionarily conserved function of a viral microRNA. J. Virol. 82: 9823-9828
Weber, K., M. E. Bolander, and G. Sarkar. 1998. PIG-B: a homemade monophasic cocktail for the extraction of RNA Mol. Biotechnol. 9: 73-77.
Théry, C., A. Clayton, S. Amigorena, and G. Raposo. 2006. Curr. Prot. in Cell Biol. 3.22.1-3.22.29.
M Bakhti, C Winter, M Simons. 2011. Journal of Biological Chemistry, 286(1):787-96.

### VI. Embodiments

### Embodiment 1

A method of detecting a glia derived extracellular JCV miRNA in a sample derived from a subject, said method comprising:

(i) isolating a glia derived extracellular JCV miRNA within a sample derived from a subject thereby producing isolated glia derived extracellular JCV miRNA;
(ii) reverse transcribing said isolated glia derived extracellular JCV miRNA thereby producing glia derived JCV cDNA;
(iii) amplifying said glia derived JCV cDNA thereby forming a plurality of amplified glia derived JCV cDNAs and a plurality of complementary glia derived JCV cDNAs; and
(iv) detecting the presence of said plurality of amplified extracellular glia derived JCV cDNAs or said plurality of complementary glia derived JCV cDNAs thereby detecting said glia derived extracellular JCV miRNA in said sample.

### Embodiment 2

The method of embodiment 1 wherein detecting said glia-derived extracellular JCV-miRNA in said sample indicates detecting a JCV-infected glia within said subject.

### Embodiment 3

The method of embodiment 1 or 2, wherein said detecting comprises:

(a) determining an amount of said plurality of amplified glia-derived extracellular JCV-cDNAs or said plurality of complementary glia-derived extracellular JCV-cDNAs;
(b) based on said amount, determining a level of glia-derived extracellular JCV-miRNAs within said sample; and
(c) comparing said level to a standard control level, wherein said level being higher than said standard control level is indicative of said subject having PML.

### Embodiment 4

The method of one of embodiments 1 to 3, wherein said subject is a mammalian subject.

### Embodiment 5

The method of one of embodiments 1 to 4, wherein said subject is a human subject.

### Embodiment 6

The method of one of embodiments 1 to 5, wherein said subject is a PML patient.

### Embodiment 7

The method of one of embodiments 1 to 6, wherein said sample is a fluid sample.

### Embodiment 8

The method of one of embodiments 1 to 7, wherein said sample is a blood sample or a urine sample.

### Embodiment 9

The method of embodiment 8, wherein said blood sample is a blood plasma sample.

### Embodiment 10

The method of one of embodiments 8 or 9, wherein said blood sample is a blood serum sample.

### Embodiment 11

The method of any one of embodiments 1 to 10, wherein said isolating comprises centrifuging said sample under conditions suitable to form an enriched glia-derived extracellular JCV-miRNA sample fraction.

### Embodiment 12

The method of any one of embodiments 1 to 10, wherein said glia-derived extracellular JCV-miRNA within said sample forms part of a JCV-miRNA infected glia-derived exosome.

### Embodiment 13

The method of embodiment 12, wherein said isolating comprises separating said JCV-miRNA infected glia-derived exosome from components of said sample.

### Embodiment 14

The method of embodiment 13, wherein said separating comprises contacting said JCV-miRNA infected glia-derived exosome with a glia-derived exosome-binding protein to form an glia-derived exosome-binding protein complex and isolating said exosome-binding protein complex.

### Embodiment 15

The method of embodiment 14, wherein said glia-derived exosome-binding protein is an glia-derived exosome binding antibody.

### Embodiment 16

The method of embodiment 1, wherein said reverse transcribing comprises contacting said isolated glia-derived extracellular JCV-miRNA with a JCV-miRNA nucleic acid probe, a reverse transcriptase and nucleic acid monomers.

### Embodiment 17

An isolated glia-derived extracellular JCV-miRNA.

### Embodiment 18

The isolated glia-derived extracellular JCV-miRNA of embodiment 17, wherein said isolated glia-derived extracellular JCV-miRNA forms part of a JCV-miRNA infected glia exosome.

### Embodiment 19

A JCV-miRNA infected glia-derived exosome.

What is claimed is:

1. A method of detecting a glia-derived extracellular JCV-miRNA in a sample derived from a subject, said method comprising:
(i) isolating a glia-derived extracellular JCV-miRNA within a sample derived from a subject thereby producing isolated glia-derived extracellular JCV-miRNA;
(ii) reverse transcribing said isolated glia-derived extracellular JCV-miRNA thereby producing glia-derived JCV-cDNA;
(iii) amplifying said glia-derived JCV-cDNA thereby forming a plurality of amplified glia-derived JCV-cDNAs and a plurality of complementary glia-derived JCV-cDNAs; and
(iv) detecting the presence of said plurality of amplified extracellular glia-derived JCV-cDNAs or said plurality of complementary glia-derived JCV-cDNAs thereby detecting said glia-derived extracellular JCV-miRNA in said sample.

2. The method of claim 1 wherein detecting said glia-derived extracellular JCV-miRNA in said sample indicates detecting a JCV-infected glia within said subject.

3. The method of claim 1, wherein said detecting comprises:
(a) determining an amount of said plurality of amplified glia-derived extracellular JCV-cDNAs or said plurality of complementary glia-derived extracellular JCV-cDNAs;
(b) based on said amount, determining a level of glia-derived extracellular JCV-miRNAs within said sample; and
(c) comparing said level to a standard control level, wherein said level being higher than said standard control level is indicative of said subject having PML.

4. The method of one of claim 1, wherein said subject is a mammalian subject.

5. The method of one of claim 1, wherein said subject is a human subject.

6. The method of one of claim 1, wherein said subject is a PML patient.

7. The method of one of claim 1, wherein said sample is a fluid sample.

8. The method of one of claim 1, wherein said sample is a blood sample or a urine sample.

9. The method of claim 8, wherein said blood sample is a blood plasma sample.

10. The method of one of claim 8, wherein said blood sample is a blood serum sample.

11. The method of any one of claim 1, wherein said isolating comprises centrifuging said sample under conditions suitable to form an enriched glia-derived extracellular JCV-miRNA sample fraction.

12. The method of any one of claim 1, wherein said glia-derived extracellular JCV-miRNA within said sample forms part of a JCV-miRNA infected glia-derived exosome.

13. The method of claim 12, wherein said isolating comprises separating said JCV-miRNA infected glia-derived exosome from components of said sample.

14. The method of claim 13, wherein said separating comprises contacting said JCV-miRNA infected glia-derived exosome with a glia-derived exosome-binding protein to form an glia-derived exosome-binding protein complex and isolating said exosome-binding protein complex.

15. The method of claim 14, wherein said glia-derived exosome-binding protein is an glia-derived exosome binding antibody.

16. The method of claim 1, wherein said reverse transcribing comprises contacting said isolated glia-derived extracellular JCV-miRNA with a JCV-miRNA nucleic acid probe, a reverse transcriptase and nucleic acid monomers.

17. An isolated glia-derived extracellular JCV-miRNA.

18. The isolated glia-derived extracellular JCV-miRNA of claim 17, wherein said isolated glia-derived extracellular JCV-miRNA forms part of a JCV-miRNA infected glia exosome.

19. A JCV-miRNA infected glia-derived exosome.
